(19) United States
(12) Patent Application Publication
(10) Pub. No.: US 2002/0164297 A1
(43) Pub. Date: Nov. 7, 2002

(54) Composition cosmtique comprenant une dispersion de particles de polymere et une dispersion de pigments

(76) Inventors: Veronique Ferrari, Malsons-Alfort (FR); Rolf Klucker, Bourg la Reine (FR)

Correspondence Address: Thomas L. Irving, FINNEGAN, HENDERSON, FARABOW, GARRETT & DUNNER, L.L.P., 1300 I Street, N.W., Washington, DC, 20005-3315, US

(21) Appl. No.: 09/987,427

(22) Filed: Nov. 14, 2001

Related U.S. Application Data
(60) Provisional application No. 60/294,979, filed on Jun. 4, 2001.

(30) Foreign Application Priority Data
Nov. 15, 2000 (FR) 00 14790

(51) Int. Cl. A61K 7/06

(52) U.S. Cl. 424/70.1

(57) ABSTRACT

The present invention relates to a composition, especially a cosmetic composition, for caring for or making up the skin and/or integuments and/or the lips, which may be in the form of a cast product comprising a physiologically acceptable medium containing at least one liquid fatty phase, a colloidal dispersion of particles that are solid at ambient temperature, and especially colored, and a dispersion of polymer particles that are surface-stabilized in the said liquid fatty phase with a stabilizer. According to the amount of polymer, it is possible to obtain on the skin, integuments or the lips a supple, glossy film that covers well while at the same time is very comfortable to wear.

The invention also relates to a care process or make-up process for the skin, integuments and the lips using such a composition.

The present invention relates to a composition containing a polymer that is dispersible in a fatty phase, intended in particular for cosmetics. More especially, the invention relates to a care and/or make-up and/or treatment composition for keratin materials such as the skin both of the human face and body, including the scalp, integuments such as the eyelashes, the eyebrows, the nails and the hair, and also the lips and the lower or upper eyelids of human beings. This composition in particular gives a glossy deposit that covers well and is not sticky on keratin materials and/or the lips.

This composition can especially be in the form of a product cast as a stick or a dish, for instance lipsticks or lip balms, cast foundations, concealer products, eyeshadows or face powders, in the form of a more or less fluid paste or cream, for instance fluid foundations or lipsticks, eyeliners, mascaras, antisun compositions, skin-colouring compositions or body make-up compositions.

Make-up or care compositions for human skin or lips, for instance foundations or lipsticks, generally contain fatty phases such as waxes and oils, pigments and/or fillers and optionally additives, for instance cosmetic or dermatological active agents. They can also contain “pasty” products of soft consistency, for obtaining coloured or non-coloured pastes to be applied with a brush.

These compositions have a tendency to migrate, that is to say to travel over time into the wrinkles and fine lines of the skin that especially surround the lips and the eyes, resulting in an unattractive effect. This migration is often quoted by women as being a major defect of conventional lipsticks. The term “migration” means an overflowing of the composition, and in particular of the colour, beyond the initial line of the make-up. In addition, it shows poor staying power over time, and in particular of the colour. This poor staying power is characterized by a modification of the colour (colour change or fading of the colour) generally following an interaction with the sebum and sweat secreted by the skin in the case of foundation and face powder, or an interaction with saliva in the case of lipsticks. This poor staying power obliges the user to freshen the make-up very regularly, which may constitute wasted time.

To increase the staying power over time, the company Shiseido envisaged, in patent application JP-A-61-65809, lipstick compositions containing a siloxysilicate resin (with a three-dimensional network), a volatile silicone oil containing a cyclic silicone chain and pulverulent fillers. Although these compositions have improved staying-power properties, they have the drawback of leaving on the lips, after the silicone oils have evaporated off, a film which becomes uncomfortable over time (sensation of dryness and tautness), which puts a certain number of women off this type of lipstick.

In addition, these compositions based on silicone oils and silicone resins produce matt coloured films. However, women are nowadays looking for glossy products, especially for colouring the lips.

To overcome these drawbacks, the Applicant has envisaged the manufacture of make-up compositions containing polymer particles that are dispersed and surface-stabilized with a stabilizer in a liquid fatty phase, as described in document EP-A-930 060. Unfortunately, the Applicant was confronted with the difficulty of introducing into certain liquid fatty phases, such as hydrogenated polyisobutenes (Parleam), a large amount of solid particles, especially coloured, that are insoluble in the medium of these compositions, for instance pigments and nacres.

Specifically, the inventors have found that above a small percentage of solid particles, these particles flocculate and aggregate or interact with the stabilizer of the polymer particles, thus rapidly destabilizing the compositions during their manufacture. This is a particular nuisance when the composition is in solid form, since it then has a non-uniform appearance in terms of colour. White lines are especially observed. This is a particular nuisance when the composition is a make-up product. Specifically, the make-up effect obtained is non-uniform and unattractive, and accentuates the defects of the support and especially of the skin, which is entirely the opposite of what the consumers want.

The subject of the invention is, precisely, a care and/or make-up and/or treatment composition for keratin materials such as the skin and/or integuments and also for the lips of the face, which allows the various drawbacks mentioned above to be overcome.

In particular, the composition of the invention produces a deposit that is not sticky, gives good coverage with a more or less glossy appearance, which is what consumers want, does not migrate, has good staying power and does not dry out the skin or the lips onto which it is applied, either during the application or over time. It also has good stability properties and, when it is in stick form, limits the heterogeneity of colour. It thus gives a uniform and attractive make-up effect.

The invention applies not only to make-up products for the lips, but also to care and/or treatment products for the lips, for instance balms, for the skin, including the scalp, for instance daily care creams and antisun creams for facial skin, make-up products for the skin, both of the human face and body, for instance foundations that are especially cast as a stick or a dish, concealer products and temporary tattoo products, body hygiene products, for instance deodorants especially in stick form, and make-up products for the eyes, for instance eyeliners, in particular in pencil form, and mascaras, especially in cake form.

The Applicant has found, entirely surprisingly, that the use of a polymer that is dispersible in a fatty phase, combined with a colloidal dispersion of solid particles that are especially coloured, in a composition, especially a cosmetic composition, produces a glossy film with very good staying power and a uniform colour, which does not migrate and is waterproof, while at the same time being very pleasant to apply and to wear throughout the day. The film is especially supple, flexible and uniform.

One subject of the present invention is thus a composition for application to keratin materials, comprising a physiologically acceptable medium containing at least one liquid fatty phase, a colloidal dispersion of particles that are solid at ambient temperature and a dispersion of polymer particles that are surface-stabilized in the said liquid fatty phase with a stabilizer.

For the purposes of the application, the expression “liquid fatty phase” means any non-aqueous medium that is liquid at ambient temperature (25° C.) and atmospheric pressure (760 mm Hg), composed of one or more fatty substances that are liquid at ambient temperature, also known as oils. This fatty phase is macroscopically homogeneous (that is to say homogeneous to the naked eye). This fatty phase may contain a volatile liquid fatty phase and/or a non-volatile fatty phase.

The expression “non-volatile fatty phase” means any medium that is liable to remain on the skin or the lips for several hours. A non-volatile fatty phase in particular has a non-zero vapour pressure at ambient temperature and atmospheric pressure, of less than 0.02 mm Hg and better still less than 10⁻³ mm Hg.

The expression “volatile fatty phase” means any non-aqueous medium capable of evaporating from the skin or the lips in less than one hour at ambient temperature and atmospheric pressure. This volatile phase especially comprises oils having a vapour pressure, at ambient temperature (25° C.) and atmospheric pressure (760 mm Hg) ranging from 10⁻³ to 300 mm Hg (0.13 Pa to 40 000 Pa) and in particular ranging from 0.02 to 300 mm Hg (2.66 Pa to 40 000 Pa).

This composition is, in particular, a cosmetic, dermatological, hygiene or pharmaceutical composition. It thus contains ingredients that are compatible with the skin, the lips and integuments, for instance keratin fibres. It may be in the form of an anhydrous gel, an oil-in-water or water-in-oil emulsion or dispersion, or alternatively in the form of a multiple emulsion. It may be in the form of a paste, a more or less rigid solid, a cream or an ointment. Preferably, it is in anhydrous form, and more especially in the form of an anhydrous gel, especially cast as a stick or a dish.

According to the invention, this composition is stable over time and temperature-stable. In particular, no segregation of the dispersed solid particles and no phase separation is observed, even after 2 months at ambient temperature and atmospheric pressure and at 45° C. In addition, when it is in stick form, its appearance to the naked eye is uniform and free of white lines.

According to the invention, the polymer is a solid that is insoluble in the fatty phase even at its softening point, unlike a wax even of polymeric origin, which is soluble in the fatty phase at its melting point. It also allows the formation of a deposit, especially a continuous and uniform film-forming deposit, and/or is characterized by the interlocking of the polymer chains. With a wax, even one obtained by polymerization, recrystallization is obtained after melting in the fatty phase. This recrystallization is in particular responsible for the loss of gloss of the composition.

The polymer in dispersion used in the present invention may be of any nature. It is thus possible to use a free-radical polymer, a polycondensate or even a polymer of natural origin, and mixtures thereof. This polymer may be chosen by a person skilled in the art as a function of its properties and depending on the desired subsequent use for the composition. Preferably, the polymer used is film-forming, that is to say capable of forming an isolable film, alone or in combination with a plasticizer. However, it is possible to use a non-film-forming polymer.

The expression “non-film-forming polymer” means a polymer that is not capable by itself of forming an isolable film. This polymer, in combination with a non-volatile compound of the oil type, gives a continuous and uniform deposit on the skin and/or the lips.

Advantageously, the composition contains at least one ingredient chosen from cosmetic, dermatological, hygiene and pharmaceutical active agents, and dyestuffs, and mixtures thereof. By virtue of the dispersion of surface-stabilized polymer particles present in the liquid fatty phase, the composition of the invention makes it possible to keep these active agents and/or dyestuffs in the place they were deposited.

According to the invention, the amount of polymer in dispersion should be sufficient to form on the skin and/or the lips and/or the keratin fibres a film capable of trapping the dyestuffs and/or cosmetic or dermatological active agents and/or oils in order to limit their migration and to increase the staying power of the composition. The amount of polymer depends on the amount of dyestuffs and/or active agents and/or oils contained in the composition. In practice, the amount of polymer may be greater than 2% by weight (as active material) relative to the total weight of the composition.

Another subject of the invention is the use, in a composition for application to the skin, the lips and integuments, and more especially the cosmetic or hygiene use, or for manufacturing a dermatological or pharmaceutical composition for application to the skin, the lips and integuments, of particles of at least one polymer that are dispersed in a liquid fatty phase and surface-stabilized with a stabilizer, and of a colloidal dispersion of particles that are solid at ambient temperature, and especially coloured, to limit the migration of the composition and/or to increase its staying power over time and/or to obtain a uniform make-up effect and/or to conserve the gloss of the said composition.

A subject of the invention is also a cosmetic care process or make-up process for keratin materials and especially the lips, integuments or the skin, which consists in applying a cosmetic composition as defined above to the lips, integuments or the skin, respectively.

A subject of the invention is also a process for limiting the migration of a make-up composition or care composition for the skin or the lips and/or to increase the staying power over time of this composition and/or its stability, containing a liquid fatty phase and at least one ingredient chosen from dyestuffs in the form of coloured particles that are solid at ambient temperature, which consists in introducing into the liquid fatty phase polymer particles that are dispersible in the liquid fatty phase and able to be surface-stabilized with a stabilizer, and in introducing into the said liquid fatty phase the said coloured particles in the form of a colloidal dispersion.

A subject of the invention is also a process for manufacturing a stable composition for application to keratin materials, which consists in introducing into a physiologically acceptable liquid medium a) a dispersion of polymer particles that are surface-stabilized in a liquid fatty phase with a stabilizer, b) a colloidal dispersion of particles that are solid at ambient temperature, chosen from pigments, nacres and fillers and mixtures thereof, and in mixing the said medium to which is added the said dispersions a) and b).

A subject of the invention is also the use of a colloidal dispersion of particles that are solid at ambient temperature, chosen from pigments, nacres and fillers, and mixtures thereof, in a composition for application to keratin materials, containing a dispersion of polymer particles that are surface-stabilized in a liquid fatty phase with a stabilizer, to stabilize the said composition and/or to give it a uniform appearance.

One advantage of using a dispersion of polymer particles in a composition of the invention is that these particles remain in the form of elementary particles, without forming aggregates, in the fatty phase. Another advantage of the polymer dispersion is the possibility of obtaining very fluid compositions (of the order of 130 centipoises), even in the presence of a high content of polymer.

Yet another advantage of such a polymer dispersion is that it is possible to calibrate the size of the polymer particles as desired, and to modify their size “polydispersity” during the synthesis. It is thus possible to obtain particles of very small size, which are invisible to the naked eye when they are in the composition and when they are applied to the skin or the lips.

It has moreover been found that the composition according to the invention has particularly advantageous qualities of spreading on and adhesion to the skin, the lips, the eyelashes or mucous membranes, and also a pleasant, creamy feel. This composition also has the advantage of being easy to remove, especially with a standard make-up-removing milk.

Polymers in Dispersion

The composition according to the invention thus advantageously comprises one or more stable dispersions of polymer particles, that are generally spherical, of one or more polymers, in a physiologically acceptable liquid fatty phase. These dispersions may especially be in the form of nanoparticles of polymers in stable dispersion in the said fatty phase. The nanoparticles are preferably between 5 nm and 600 nm in size. However, it is possible to obtain polymer particles ranging up to 1 μm in size.

Yet another advantage of the polymer dispersion of the composition of the invention is the possibility of varying the glass transition temperature (Tg) of the polymer or the polymer system (polymer plus additive of the plasticizer type), and of thus going from a hard polymer to a more or less soft polymer, making it possible to adjust the mechanical properties of the composition depending on the intended application and in particular on the film deposited.

The polymers in dispersion which may be used in the composition of the invention preferably have a molecular weight of about from 2 000 to 10 000 000 and a Tg of from −100° C. to 300° C. and better still from −50° C. to 50° C. and preferably from −10° C. to 100° C.

When the polymer has a glass transition temperature that is too high for the desired use, a plasticizer may be combined therewith so as to lower this temperature of the mixture used. The plasticizer may be chosen from the plasticizers usually used in the field of application, and especially from compounds which may be solvents for the polymer.

It is possible to use film-forming polymers, that preferably have a low Tg, of less than or equal to the temperature of the skin and especially less than or equal to 40° C. A dispersion is thus obtained which can form a film when it is applied to a support, which is not the case when dispersions of mineral pigments according to the prior art are used.

Among the film-forming polymers which may be mentioned are free-radical, acrylic or vinyl homopolymers or copolymers, preferably having a Tg of less than or equal to 40° C. and especially ranging from −10° C. to 30° C., used alone or as a mixture.

Among the non-film-forming polymers which may be mentioned are free-radical, vinyl or acrylic homopolymers or copolymers, that are optionally crosslinked, preferably having a Tg of greater than 40° C. and especially ranging from 45° C. to 150° C., used alone or as a mixture.

The expression “free-radical polymer” means a polymer obtained by polymerization of monomers containing unsaturation, especially ethylenic unsaturation, each monomer being capable of homopolymerizing (unlike polycondensates). The free-radical polymers may especially be vinyl polymers or copolymers, especially acrylic polymers.

The vinyl polymers may result from the polymerization of ethylenically unsaturated monomers containing at least one acid group and/or esters of these acidic monomers and/or amides of these acids.

As monomers bearing an acidic group, it is possible to use α,β-ethylenic unsaturated carboxylic acids such as acrylic acid, methacrylic acid, crotonic acid, maleic acid or itaconic acid. (Meth)acrylic acid and crotonic acid are preferably used, and more preferentially (meth)acrylic acid.

The esters of acidic monomers are advantageously chosen from the esters of (meth)acrylic acid (also known as (meth)acrylates), for instance alkyl (meth)acrylates, in particular of a C₁-C₂₀ and preferably a C₁-C₈ alkyl, aryl (meth)acrylates, in particular of a C₆-C₁₀ aryl, and hydroxyalkyl (meth)acrylates, in particular of a C₂-C₆ hydroxyalkyl. Alkyl (meth)acrylates which may be mentioned include methyl, ethyl, butyl, isobutyl, 2-ethylhexyl and lauryl (meth)acrylate. Hydroxyalkyl (meth)acrylates which may be mentioned include hydroxyethyl (meth)acrylate and 2-hydroxypropyl (meth)acrylate. Aryl (meth)acrylates which may be mentioned include benzyl or phenyl acrylate.

The (meth)acrylic acid esters that are particularly preferred are the alkyl (meth)acrylates.

Free-radical polymers that are preferably used are copolymers of (meth)acrylic acid and of an alkyl (meth)acrylate, especially of a C₁-C₄ alkyl. More preferentially, methyl acrylates may be used, optionally copolymerized with acrylic acid.

Amides of the acidic monomers which may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide; N-di(C₁-C₄)alkyl(meth)acrylamides.

The vinyl polymers may also result from the polymerization of ethylenically unsaturated monomers containing at least one amine group, in free form or partially or totally neutralized, or alternatively partially or totally quaternized. Such monomers may be, for example, dimethylaminoethyl (meth)acrylate, dimethylaminoethylmethacrylamide, vinylamine, vinylpyridine and diallyldimethylammonium chloride.

The vinyl polymers may also result from the homopolymerization or copolymerization of at least one monomer chosen from vinyl esters and styrene monomers. In particular, these monomers may be polymerized with acidic monomers and/or esters thereof and/or amides thereof, such as those mentioned above. Examples of vinyl esters which may be mentioned include vinyl acetate, vinyl propionate, vinyl neodecanoate, vinyl pivalate, vinyl benzoate and vinyl t-butylbenzoate. Styrene monomers which may be mentioned include styrene and α-methylstyrene.

The list of monomers given is not limiting, and it is possible to use any monomer known to those skilled in the art which falls within the categories of acrylic and vinyl monomers (including monomers modified with a silicone chain).

As other vinyl monomers that may be used, mention may also be made of:

N-vinylpyrrolidone, vinylcaprolactam, vinyl-N-(C₁-C₆)alkylpyrroles, vinyloxazoles, vinylthiazoles, vinylpyrimidines and vinylimidazoles,

olefins such as ethylene, propylene, butylene, isoprene and butadiene.

The vinyl polymer may be crosslinked with one or more difunctional monomers, especially comprising at least two ethylenic unsaturations, such as ethylene glycol dimethacrylate or diallyl phthalate.

In a non-limiting manner, the polymers in dispersion of the invention may be chosen from the following polymers or copolymers: polyurethanes, polyurethane-acrylics, polyureas, polyurea-polyurethanes, polyester-polyurethanes, polyether-polyurethanes, polyesters, polyesteramides, fatty-chain polyesters, alkyds; acrylic and/or vinyl polymers or copolymers; acrylic-silicone copolymers; polyacrylamides; silicone polymers, for instance silicone polyurethanes or silicone acrylics, and fluoro polymers and mixtures thereof.

The polymer(s) in oily dispersion may represent (as solids or active material) from 0.1% to 60% of the weight of the composition, preferably from 2% to 40% and better still from 4% to 25%. For a stabilizer that is solid at ambient temperature, the amount of solids in the dispersion represents the total amount of polymer+stabilizer.

Fatty Phase

The liquid fatty phase of the composition may consist of any oil that is cosmetically or dermatologically acceptable and, in general, physiologically acceptable, chosen especially from oils of mineral, animal, plant or synthetic origin, carbon-based, hydrocarbon-based, fluoro and/or silicone oils, alone or as a mixture, provided that they form a macroscopically stable and homogeneous mixture and provided that they are compatible with the intended use.

The expression “hydrocarbon-based oil” means oils mainly containing carbon atoms and hydrogen atoms and in particular alkyl or alkenyl chains, for instance alkanes or alkenes, but also oils containing an alkyl or alkenyl chain comprising one or more ether, ester or carboxylic acid groups.

The total liquid fatty phase of the composition may represent from 5% to 99.40% of the total weight of the composition and preferably from 20% to 85%. It advantageously represents at least 30% of the total weight of the composition. Preferably, this fatty phase contains at least one non-volatile oil (i.e. several non-volatile oils). The non-volatile portion may represent from 0% to 80% of the total weight of the composition and better still from 1% to 50%.

As liquid fatty phases that may be used in the invention, mention may thus be made of hydrocarbon-based oils of mineral or synthetic origin, such as volatile or non-volatile linear or branched hydrocarbons, for instance liquid paraffin or its derivatives, liquid petroleum jelly, polydecenes, hydrogenated polyisobutene such as Parleam, and squalane; oils of animal origin, for instance mink oil, turtle oil or perhydrosqualene; oils of plant origin; hydrocarbon-based oils with a high triglyceride content consisting of fatty acid esters of glycerol, in which the fatty acids may have varied chain lengths, these chains possibly being linear or branched, and saturated or unsaturated, for instance sweet almond oil, beauty-leaf oil, palm oil, grapeseed oil, sesame oil, arara oil, rapeseed oil, sunflower oil, cottonseed oil, apricot oil, castor oil, alfalfa oil, marrow oil, blackcurrant oil, macadamia oil, muscat rose oil, hazelnut oil, avocado oil, jojoba oil, olive oil or cereal (corn, wheat, barley or rye) germ oil; fatty acid esters and especially esters of lanolic acid, of oleic acid, of lauric acid or of stearic acid; synthetic esters of formula R₁COOR₂ in which R₁ represents a linear or branched higher fatty acid residue containing from 7 to 40 carbon atoms and R₂ represents a branched hydrocarbon-based chain containing from 3 to 40 carbon atoms, such as, for example, purcellin oil (cetostearyl octanoate), isononyl isononanoate, C₁₂ to C₁₅ alkyl benzoate, 2-ethylhexyl palmitate, alkyl or polyalkyl octanoates, decanoates or ricinoleates, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate, glyceryl triisostearate or diglyceryl triisostearate; hydroxylated esters, for instance isostearyl lactate or diisostearyl malate; pentaerythritol esters; C₈-C₂₆ higher fatty acids such as myristic acid, palmitic acid, stearic acid, behenic acid, oleic acid, linoleic acid, linolenic acid or isostearic acid; C₈-C₂₆ higher fatty alcohols such as stearyl alcohol, oleyl alcohol, linoleyl alcohol, linolenyl alcohol, isostearyl alcohol or octyldodecanol; synthetic ethers containing at least 7 carbon atoms, silicone oils such as linear or cyclic, volatile or non-volatile polydimethylsiloxanes (PDMSs) that are liquid at room temperature, and that are optionally phenylated, such as phenyltrimethicones, phenyltrimethylsiloxydiphenylsiloxanes, diphenyldimethicones, diphenylmethyldiphenyltrisiloxanes, liquid 2-phenylethyl trimethylsiloxysilicates, optionally substituted with aliphatic and/or aromatic groups, for instance alkyl, alkoxy or phenyl groups, pendent and/or at the end of the silicone chain, these groups containing from 2 to 24 carbon atoms and being optionally fluorinated, or with functional groups such as hydroxyl, thiol and/or amine groups; polysiloxanes modified with fatty acids, fatty alcohols or polyoxyalkylenes; fluorosilicones; or alternatively caprylic/capric acid triglycerides, for instance those sold by the company Stearineries Dubois or those sold under the names Miglyol 810, 812 and 818 by the company Dynamit Nobel; and mixtures thereof.

One or more solvents that are volatile at ambient temperature may advantageously be used as volatile cosmetic oils. These volatile solvents are favourable towards the production of a deposit of good staying power. After these solvents have evaporated off, a supple film-forming deposit that is not sticky on the skin or the lips is obtained. These volatile solvents also facilitate the application of the composition to the skin, the lips and integuments. They may be hydrocarbon-based or silicone-based and may optionally comprise alkyl or alkoxy groups that are pendent or at the end of the silicone chain.

As volatile oils that may be used in the invention, mention may be made of linear or cyclic silicone oils having a viscosity at ambient temperature of less than 8 cSt and especially containing from 2 to 7 silicon atoms, these silicones optionally comprising alkyl or alkoxy groups containing from 1 to 10 carbon atoms. As volatile silicone oils that may be used in the invention, mention may especially be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As other volatile solvents that may be used in the invention, mention may be made of hydrocarbon-based volatile oils containing from 8 to 16 carbon atoms and mixtures thereof, and especially branched C₈-C₁₆ alkanes, for instance C₈-C₁₆ isoalkanes (also known as isoparaffins), isododecane, isodecane and isohexadecane and, for example, the oils sold under the trade names “Isopars” or “Permetyls”, and C₈-C₁₆ branched esters, for instance isohexyl neopentanoate, and the mixtures thereof.

In one particular embodiment of the invention, the liquid fatty phase is chosen from the group comprising:

non-aqueous liquid compounds having a global solubility parameter according to the Hansen solubility space of less than 17 (MPa)^{½}.

or monoalcohols having a global solubility parameter according to the Hansen solubility space of less than or equal to 20 (MPa)^{½},

or mixtures thereof.

The global solubility parameter δ global according to the Hansen solubility space is defined in the article “Solubility parameter values” by Eric A. Grulke in the book “Polymer Handbook” 3rd Edition, Chapter VII, pages 519-559, by the relationship:

δ=(d_{D}²+d_{P}²+d_{H}²)^{½}

in which

d_{D} characterizes the London dispersion forces arising from the formation of dipoles induced during molecular impacts,

d_{P} characterizes the Debye interaction forces between permanent dipoles,

d_{H} characterizes the forces of specific interactions (such as hydrogen bonding, acid/base, donor/acceptor, etc.). The definition of solvents in the three-dimensional solubility space according to Hansen is described in the article by C. M. Hansen: “The three-dimensional solubility parameters”, J. Paint Technol. 39, 105 (1967).

Among the liquid fatty phases having a global solubility parameter according to the Hansen solubility space of less than or equal to 17 (MPa )^{½}, mention may be made of plant oils formed from fatty acid esters and from polyols, in particular triglycerides, such as sunflower oil, sesame oil or rapeseed oil, or esters derived from acids or alcohols containing a long chain (i.e. a chain containing from 6 to 20 carbon atoms), in particular the esters of formula R₁COOR₂ in which R₁ represents a higher fatty acid residue containing from 7 to 40 carbon atoms and R₂ represents a hydrocarbon-based chain containing from 3 to 40 carbon atoms, such as palmitates, adipates and benzoates, in particular diisopropyl adipate. Mention may also be made of hydrocarbons and in particular liquid paraffin, liquid petroleum jelly or hydrogenated polyisobutylene, isododecane or “Isopars” (volatile isoparaffins). Mention may also be made of silicone oils such as polydimethylsiloxanes and polymethylphenylsiloxanes, optionally substituted with aliphatic and/or aromatic groups, optionally fluorinated, or optionally substituted with functional groups such as hydroxyl, thiol and/or amine groups, and volatile silicone oils, in particular cyclic volatile silicone oils. Mention may also be made of solvents, alone or as a mixture, chosen from (i) linear, branched or cyclic esters containing more than 6 carbon atoms, (ii) ethers containing more than 6 carbon atoms, (iii) ketones containing more than 6 carbon atoms. The expression “monoalcohols having a global solubility parameter according to the Hansen solubility space of less than or equal to 20 (MPa)^{½}” means aliphatic fatty alcohols containing at least 6 carbon atoms, the hydrocarbon-based chain containing no substituent groups. Monoalcohols according to the invention which may be mentioned are oleyl alcohol, decanol, dodecanol, octadecanol and linoleyl alcohol.

Non-aqueous media which can also be used are those described in document FR-A-2 710 646 from L.V.M.H.

The choice of the non-aqueous medium is made by a person skilled in the art on the basis of the nature of the monomers constituting the polymers and/or the nature of the stabilizer, as indicated below.

The polymer dispersion may be manufactured as described in document EP-A-749 747. The polymerization can be carried out by dispersion, that is to say by precipitating the polymer as it forms, with protection of the particles formed with a stabilizer.

A mixture is thus prepared comprising the initial monomers as well as a radical initiator. This mixture is dissolved in a solvent, which is referred to in the rest of the present description as the “synthesis solvent”. When the fatty phase is a non-volatile oil, the polymerization can be carried out in an apolar organic solvent (synthesis solvent) followed by addition of the non-volatile oil (which should be miscible with the said synthesis solvent) and selective distillation of the synthesis solvent.

A synthesis solvent is thus chosen such that the initial monomers and the radical initiator are soluble therein, and the particles of each polymer obtained are insoluble therein, in order for them to precipitate therein as they are formed. In particular, the synthesis solvent can be chosen from alkanes such as heptane, isododecane or cyclohexane.

When the fatty phase chosen is a volatile oil, the polymerization of each polymer can be carried out directly in the said oil which thus also acts as synthesis solvent. The monomers should also be soluble therein, as should the radical initiator, and the polymer obtained should be insoluble therein.

The monomers are preferably present in the synthesis solvent, before polymerization, in a proportion of 5-20% by weight of the reaction mixture. The total amount of the monomers can be present in the solvent before the start of the reaction, or some of the monomers can be added gradually as the polymerization reaction proceeds.

The radical initiator can be, in particular, azobis(isobutyronitrile) or tert-butylperoxy-2-ethyl hexanoate.

The Stabilizer:

The polymer particles are surface-stabilized, gradually as the polymerization proceeds, by means of a stabilizer which can be a block polymer, a grafted polymer, and/or a random polymer, alone or as a mixture. The stabilization can be carried out by any known means, and in particular by direct addition of the block polymer, grafted polymer and/or random polymer during the polymerization.

The stabilizer is preferably also present in the mixture before polymerization. However, it is also possible to add it continuously, in particular when the monomers are also added continuously.

2-30% by weight of stabilizer can be used relative to the initial monomer mixture, and preferably 5-20% by weight.

When a grafted and/or block polymer is used as stabilizer, the synthesis solvent is chosen such that at least some of the grafts or blocks of the said stabilizing polymer are soluble in the said solvent, the rest of the grafts or blocks being insoluble therein. The stabilizing polymer used during the polymerization should be soluble, or dispersible, in the synthesis solvent. Furthermore, a stabilizer whose insoluble blocks or grafts have a certain affinity for the polymer formed during the polymerization is preferably chosen.

Among the grafted polymers which may be mentioned are silicone polymers grafted with a hydrocarbon-based chain; hydrocarbon-based polymers grafted with a silicone chain.

Grafted copolymers having, for example, an insoluble skeleton of polyacrylic type with soluble grafts of poly(12-hydroxystearic acid) type are also suitable for use.

Thus, grafted-block or block copolymers comprising at least one block of polyorganosiloxane type and at least one block of a free-radical polymer can be used, such as grafted copolymers of acrylic/silicone type which can be used in particular when the non-aqueous medium is a silicone medium.

Grafted-block or block copolymers comprising at least one block of polyorganosiloxane type and at least one block of polyether type may also be used. The polyorganopolysiloxane block can be, in particular, a polydimethylsiloxane or a poly(C₂-C₁₈)alkylmethylsiloxane; the polyether block can be a poly(C₂-C₁₈)alkylene, in particular polyoxyethylene and/or polyoxypropylene. In particular, dimethicone copolyols or (C₂-C₁₈)alkylmethicone copolyols can be used. It is possible, for example, to use the dimethicone copolyol sold under the name “Dow Corning 3225C” by the company Dow Corning, or the lauryl methicone copolyol sold under the name “Dow Corning Q2-5200” by the company Dow Corning.

Grafted-block or block copolymers which can also be used are copolymers comprising at least one block resulting from the polymerization of at least one ethylenic monomer, containing one or more optionally conjugated ethylenic bonds, such as ethylene, butadiene or isoprene, and of at least one block of a vinyl, or preferably styrene, polymer. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of ethylenepropylene blocks, and the polymerization of butadiene leads, after hydrogenation, to the formation of ethylene-butylene blocks. Among these block copolymers which may be mentioned are copolymers of “diblock” or “triblock” type such as polystyrene/polyisoprene (SI) or polystyrene/polybutadiene (SB), such as those sold under the name ‘Luvitol HSB’ by BASF, of the polystyrene/copoly(ethylene-propylene) (SEP) type, such as those sold under the name ‘Kraton’ by Shell Chemical Co. or alternatively of the polystyrene/copoly(ethylene-butylene) (SEB) type. In particular, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) or Kraton D-1107 (SIS) may be used. Polymers are generally known as hydrogenated or non-hydrogenated diene copolymers.

Gelled Permethyl 99A-750, 99A-753-59 and 99A-753-58 (mixture of triblock and starburst polymer), Versagel 5960 from Penreco (triblock+starburst polymer); OS129880, OS129881 and OS84383 from Lubrizol (styrene/methacrylate copolymer) may also be used.

As grafted-block or block copolymers comprising at least one block resulting from the polymerization of at least one ethylenic monomer with one or more ethylenic bonds, and of at least one block of an acrylic polymer, mention may be made of poly(methyl methacrylate)/polyisobutylene diblock or triblock copolymers or grafted copolymers with a poly(methyl methacrylate) skeleton and with polyisobutylene grafts.

As grafted-block or block copolymers comprising at least one block resulting from the polymerization of at least one ethylenic monomer with one or more ethylenic bonds and of at least one block of a polyether such as a C₂-C₁₈ polyalkylene, in particular polyethylene and/or polyoxypropylene, mention may be made of polyoxyethylene/polybutadiene or polyoxyethylene/polyisobutylene diblock or triblock copolymers.

When a random polymer is used as stabilizer, it is chosen such that it has a sufficient amount of groups that make it soluble in the intended synthesis solvent.

Copolymers based on acrylates or methacrylates of alkyls derived from C₁-C₄ alcohols, and acrylates or methacrylates of alkyls derived from C₈-C₃₀ alcohols may thus be used. Mention may be made in particular of the stearyl methacrylate/methyl methacrylate copolymer.

When the synthesis solvent is apolar, the stabilizer preferably chosen is a polymer which covers the particles as completely as possible, several stabilizing-polymer chains then becoming adsorbed on a polymer particle obtained by polymerization.

In this case, the stabilizer preferably used is either a grafted polymer or a block polymer, so as to have better interfacial activity. The reason for this is that the blocks or grafts that are insoluble in the synthesis solvent provide more voluminous coverage at the surface of the particles.

When the liquid synthesis solvent comprises at least one silicone oil, the stabilizer is preferably chosen from the group consisting of grafted-block or block copolymers comprising at least one block of polyorganosiloxane type and at least one block of a free-radical polymer or of a polyether or a polyester, such as polyoxypropylenated and/or polyoxyethylenated blocks.

When the liquid fatty phase does not comprise a silicone oil, the stabilizer is preferably chosen from the group consisting of:

(a) grafted-block or block copolymers comprising at least one block of polyorganosiloxane type and at least one block of a free-radical polymer or of a polyether or a polyester,

(b) copolymers of C₁-C₄ alkyl acrylates or methacrylates and of C₈-C₃₀ alkyl acrylates or methacrylates,

(c) grafted-block or block copolymers comprising at least one block resulting from the polymerization of at least one ethylenic monomer containing conjugated ethylenic bonds,

and at least one block of a vinyl or acrylic polymer or of a polyether or a polyester, or mixtures thereof.

Diblock polymers are preferably used as stabilizer.

The gelled polymer dispersions obtained according to the invention may then be used in a composition, especially a cosmetic, dermatological, pharmaceutical and/or hygiene composition, such as a care or make-up composition for the skin or the lips, or alternatively a hair composition or an antisun composition or a skin-colouring or artificial-tanning composition.

Depending on the application, it may be chosen to use dispersions of film-forming or non-film-forming polymers in volatile or non-volatile oils.

Colloidal Dispersion

The composition according to the invention also contains particles that are solid at ambient temperature, dispersed in the physiologically acceptable medium and introduced into the composition in the form of a colloidal dispersion, also known as a “particulate paste”.

For the purposes of the invention, the expressions “colloidal dispersion” and “particulate paste” mean a concentrated colloidal dispersion of coated or uncoated particles in a continuous medium, that is stabilized using a dispersant or optionally without a dispersant. These particles may be chosen from pigments, nacres and solid fillers, and mixtures thereof. These particles may be in any form, especially spherical or elongated form like fibres. They are insoluble in the medium.

The dispersant serves to protect the dispersed particles against their aggregation or flocculation. The dispersant concentration generally used to stabilize a colloidal dispersion is from 0.3 to 5 mg/m² and preferably from 0.5 to 4 mg/m² of surface area of particles. This dispersant may be a surfactant, an oligomer, a polymer or a mixture of several of them, bearing one or more functionalities having a strong affinity for the surface of the particles to be dispersed. In particular, they may attach physically or chemically to the surface of pigments. These dispersants also contain at least one functional group that is compatible with or soluble in the continuous medium. In particular, esters of 12-hydroxystearic acid in particular and of a C₈ to C₂₀ fatty acid and of a polyol, for instance glycerol or diglycerol, are used, such as the stearate of poly(12-hydroxystearic acid) with a molecular weight of about 750 g/mol, such as the product sold under the name Solsperse 21 000 by the company Avecia, the polyglyceryl-2 dipolyhydroxystearate (CTFA name) sold under the reference Dehymyls PGPH by the company Henkel or polyhydroxystearic acid, such as the product sold under the reference Arlacel P100 by the company Uniqema, and mixtures thereof.

As other dispersants which may be used in the composition of the invention, mention may be made of quaternary ammonium derivatives of polycondensed fatty acids, for instance Solsperse 17 000 sold by the company Avecia, and mixtures of polydimethylsiloxane/oxypropylene, such as those sold by the company Dow Corning under the references DC2-5185 and DC2-5225 C.

Polydihydroxystearic acid and the 12-hydroxystearic acid esters are preferably intended for a hydrocarbon-based or fluorinated medium, whereas the mixtures of oxyethylenated/oxypropylenated dimethylsiloxane are preferably intended for a silicone medium.

The colloidal dispersion is a suspension of particles that are generally micron-sized (<10 μm) in a continuous medium. The volume fraction of particles in a concentrated dispersion is from 20% to 40% and preferably greater than 30%, which corresponds to a weight content that may be up to 70% depending on the density of the particles.

The particles dispersed in the medium may consist of mineral or organic particles or mixtures thereof, such as those described below.

The continuous medium of the paste may be of any nature and may contain any solvent or liquid fatty substance and mixtures thereof. Advantageously, the liquid medium of the particulate paste is one of the liquid fatty substances or oils that it is desired to use in the composition, thus forming part of the liquid fatty phase.

Advantageously, the “particulate paste” or colloidal dispersion is a “pigmentary paste” containing a colloidal dispersion of coated or uncoated coloured particles. These coloured particles are pigments, nacres or a mixture of pigments and/or nacres.

Advantageously, the colloidal dispersion represents from 0.5% to 60% by weight of the composition and better still from 2% to 40% and even better still from 2% to 30%.

The pigments may be white or coloured, mineral and/or organic, and interferential or non-interferential. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue. Among the organic pigments that may be mentioned are carbon black, pigments of barium, strontium, calcium or aluminium organic lake type, including those submitted for certification by the Food and Drug Administration (FDA) (example: D&C or FD&C) and those that are exempt from FDA certification, for instance lakes based on cochineal carmine. The pigments may represent from 0.1% to 50% as active material and especially from 0.5% to 35% and better still from 2% to 25% of the total weight of the composition.

The nacreous pigments can be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the abovementioned type, as well as nacreous pigments based on bismuth oxychloride. They may represent from 0% to 25% (as active material) of the total weight of the composition and better still from 0.1% to 15% (if present). Pigments with goniochromatic properties may thus be used.

The fillers may be mineral or organic, and lamellar or spherical. Mention may be made of talc, mica, silica, kaolin, Nylon® powder (Orgasol® from Atochem), poly-β-alanine powder and polyethylene powder, lauroyllysine, starch, boron nitride, tetrafluoroethylene polymer powders (Teflon), hollow microspheres such as Expancel® (Nobel Industrie), Polytrap® (Dow Corning) and silicone resin microbeads (Tospearls® from Toshiba, for example), precipitated calcium carbonate, magnesium carbonate and hydrocarbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example zinc, magnesium or lithium stearate, zinc laurate or magnesium myristate.

The composition of the invention may also contain dyes that are soluble in the physiological medium, and in particular liposoluble or water-soluble dyes.

The liposoluble dyes are, for example, Sudan red, D&C Red No. 17, D&C Green No. 6, β-carotene, soybean oil, Sudan brown, D&C Yellow No. 11, D&C Violet No. 2, D&C Orange No. 5, quinoline yellow and bromo acids. They may represent from 0% to 20% and especially 0.01% to 20% of the weight of the composition and better still from 0.1% to 6% (if present). The water-soluble dyes are, for example, beetroot juice and methylene blue, and may represent up to 6% of the total weight of the composition.

The composition of the invention may also contain one or more cosmetic or dermatological active agents such as those conventionally used.

As cosmetic, dermatological, hygiene or pharmaceutical active agents that may be used in the composition of the invention, mention may be made of moisturizers, vitamins, essential fatty acids, sphingolipids and sunscreens. These active agents are used in the usual amount for those skilled in the art, and especially at concentrations of from 0% to 20% and especially from 0.001% to 15% of the total weight of the composition.

The polymer in the composition of the invention allows the formation of a film on the skin, the lips and/or integuments, forming a network that traps the dyestuffs (including the fillers) and/or the active agents. Depending on the relative amount of dyestuffs used relative to the amount of stabilized polymer used, it is possible to obtain a more or less glossy film that has good staying power, is water-resistant and has a uniform colour.

Depending on the type of use envisaged, the composition according to the invention may also comprise the constituents conventionally used in the fields considered, which are present in an amount that is suitable for the intended presentation form.

In particular, it can comprise, besides the liquid fatty phase in which the polymer particles are stabilized, additional fatty phases that may be chosen from waxes, oils, gums and/or pasty fatty substances, that are hydrocarbon-based, silicone-based and/or fluorinated, of plant, animal, mineral or synthetic origin, and mixtures thereof.

Among the waxes which are solid at ambient temperature, which can be present in the composition according to the invention, mention may be made of hydrocarbon-based waxes such as beeswax, carnauba wax, candelilla wax, ouricurry wax, Japan wax, cork fibre wax or sugar cane wax, paraffin wax, lignite wax, microcrystalline waxes, lanolin wax, montan wax, ozokerites, polyethylene waxes, the waxes obtained by Fischer-Tropsch synthesis, hydrogenated oils, fatty esters and glycerides which are solid at 25° C. Silicone waxes can also be used, among which mention may be made of alkyl, alkoxy and/or esters of polymethylsiloxane. The waxes can be in the form of stable dispersions of colloidal wax particles, as can be prepared according to known methods, such as those in “Microemulsions Theory and Practice”, L. M. Prince Ed, Academic Press (1977), pages 21-32. Preferably, the waxes used have a melting point at least equal to 45° C.

The waxes may be present in a proportion of 0-50% by weight in the composition and better still from 5% to 25%, so as not to excessively reduce the gloss of the composition or of the film deposited on the lips and/or the skin.

The composition may also comprise any additive usually used in such compositions, such as water, thickeners for an oil phase or for an aqueous phase, antioxidants, fragrances, preserving agents, surfactants, essential oils and liposoluble polymers (polyvinylpyrrolidone/eicosine copolymer).

In one specific embodiment of the invention, the compositions according to the invention can be prepared in the usual manner by a person skilled in the art. They can be in the form of a cast product and, for example, in the form of a stick or tube, or in the form of a dish which can be used by direct contact or with a sponge. In particular, they find an application as a cast foundation, a cast face powder or eye shadow, a lipstick, a lipcare balm or base or a concealer product. They can also be in the form of a supple paste or alternatively in the form of a gel or a more or less fluid cream. In this case, they can constitute foundations or lipsticks, antisun products or skin-colouring products.

The compositions of the invention are advantageously anhydrous and can contain less than 5% water relative to the total weight of the composition. In this case, they can in particular be in the form of an oily gel, an oily liquid, an oil, a paste or a stick, or alternatively in the form of a vesicular dispersion containing ionic and/or nonionic lipids. They may also be in the form of a simple or multiple emulsion containing an oily or aqueous continuous phase, or in the form of an oily dispersion in an aqueous phase by means of vesicles containing ionic and/or nonionic lipids. These presentation forms are prepared according to the usual methods of the fields considered.

These compositions for topical application can in particular constitute a cosmetic, dermatological, hygiene or pharmaceutical composition for protecting, treating or caring for the face, for the neck, for the hands or for the body (for example, a care cream, an antisun oil or a body gel), a make-up composition (for example, a make-up gel, a cream or a stick) or an artificial-tanning composition or protective composition for the skin.

The composition according to the invention may be in the form of a dermatological or care composition for the skin and/or integuments or in the form of an antisun composition or a body hygiene composition, especially in the form of a deodorant. In this case, it may especially be in uncoloured form. It may then be used as a care base for the skin, integuments or the lips (lip balms, for protecting the lips against the cold and/or sun and/or the wind, or a care cream for the skin, the nails or the hair).

The composition of the invention may also be in the form of a coloured product, in particular a make-up product for the skin, optionally having care or treating properties, and in particular a foundation, a blusher, a face powder, an eyeshadow, a concealer product, an eyeliner or a body make-up product; a make-up product for the lips, for instance a lipstick, a lip gloss or a lip pencil optionally having care or treating properties; a make-up product for integuments, for instance the nails, or the eyelashes in the form of a mascara, or the eyebrows and the hair.

Needless to say, the composition of the invention should be cosmetically or dermatologically acceptable, that is to say that it should contain a physiologically acceptable, non-toxic medium which may be applied to the human skin, integuments or lips of the face. For the purposes of the invention, the expression “cosmetically acceptable” means a composition of pleasant appearance, odour and feel.

Needless to say, a person skilled in the art will take care to select this or these possible additional compound(s), and/or the amount thereof, such that the advantageous properties of the composition according to the invention are not, or are not substantially, adversely affected by the addition envisaged.

The invention is illustrated in greater detail in the examples which follow. The percentages are percentages by weight.

### EXAMPLE 1 OF A POLYMER DISPERSION

A dispersion of polymethyl methacrylate crosslinked with ethylene glycol dimethacrylate, in hydrogenated polyisobutene (containing 6-8 mol of isobutylene), sold under the name Parleam by the company Nippon Oil Fat, is prepared according to the method of Example 2 of document EP-A-749 746, replacing the Isopar L with Parleam. A dispersion of polymethyl methacrylate particles that are surface-stabilized in the Parleam with a polystyrene/copoly(ethylene-propylene) diblock copolymer, sold under the name Kraton G1701 (Shell), having a solids content of 22-25% by weight and a Tg of 100° C., is thus obtained. This copolymer is not film-forming at ambient temperature.

### EXAMPLE 2 OF A POLYMER DISPERSION

A dispersion of a non-crosslinked copolymer of methyl acrylate and of acrylic acid in an 85/15 ratio, in Parleam, is prepared according to the method of Example 1 of document EP-A-749 746, replacing the heptane with Parleam. A dispersion of poly(methyl acrylate/acrylic acid) surface-stabilized particles in Parleam with a polystyrene/copoly(ethylene-propylene) diblock copolymer sold under the name Kraton G1701 (Shell), which has a solids content of 22-25% by weight and a mean particle size of 165 nm (polydispersity: 0.05) and a Tg of 13° C., is thus obtained. This copolymer can form a film.

COUNTEREXAMPLE 1
lipstick

| Phase A | |
|---|---|
| Polyethylene wax | 13% |
| Carnauba wax | 2% |
| Diisostearyl malate | 6% |
| Dispersion of Example 2 | 50% |
| Parleam | 3% |
| Phase B | |
| Lanolin | 6% |
| Pigments | 8.66% |
| Dispersant | 2% |
| Parleam | 9.34% |

All the components of phase A are introduced into a heating vessel and are heated to 100° C. with magnetic stirring so as to obtain a homogeneous mixture. Phase B is then ground in a three-roll mill. This ground material is then added to phase A. The mixture is stirred for 1 hour and then cast in suitable lipstick moulds. A stick is obtained.

When phase B is introduced (grinding of pigment in the three-roll mill), a colour change when hot is noted. Once shaped in the moulds, this formulation has the appearance of a stick that is non-uniform at the surface: presence of marbling on the stick.

EXAMPLE 3
lipstick

| Phase A | |
|---|---|
| Polyethylene wax | 13% |
| Carnauba wax | 2% |
| Diisostearyl malate | 6% |
| Dispersion of Example 2 | 50% |
| Parleam | 5.28% |
| Phase B | |
| Lanolin | 6% |
| Pigmentary paste | 17.72% |

All the components of phase A are introduced into a heating vessel and are heated to 100° C. with magnetic stirring to obtain a homogeneous mixture. Phase B corresponding to the pigmentary paste mixed with lanolin is then added. The pigmentary paste contains 8.66% pigments, 0.58% poly(12-hydroxystearic acid) stearate and 8.48% Parleam. When the pigmentary paste is introduced into phase A, it is noted that the colour change is less pronounced than in the counterexample mixture. The stick obtained shows no marbling and is uniformly coloured. It gives a deposit with good staying power on the lips, which does not migrate and feels comfortable.

COUNTEREXAMPLE 2
lipstick

| Phase A | |
|---|---|
| Polyethylene wax | 13% |
| Carnauba wax | 2% |
| Diisostearyl malate | 6% |
| Dispersion of Example 2 | 50% |
| Parleam | 9% |
| Phase B | |
| Pigments | 8.66% |
| Dispersant | 2% |
| Parleam | 9.34% |

The procedure and the appearance of the lipstick are identical to those of the lipstick of counterexample 1.

EXAMPLE 4
lipstick

| Phase A | |
|---|---|
| Polyethylene wax | 13% |
| Carnauba wax | 2% |
| Diisostearyl malate | 6% |
| Dispersion of Example 2 | 50% |
| Phase B | |
| Parleam | 11.3% |
| Pigmentary paste | 17.7% |

All the components of phase A are introduced into a heating vessel and are heated to 100° C. with magnetic stirring so as to obtain a homogeneous mixture. Phase B corresponding to the pigmentary paste mixed with Parmeam is then added. The pigmentary paste contains 8.66% pigments, 0.58% poly(12-hydroxystearic acid) stearate and 8.48% Parleam. The stick obtained shows no marbling and is uniform in colour. It is easy to apply: the deposit obtained is comfortable, uniform and glossy, does not migrate and shows good staying power, especially of the colour.

1. Composition for application to keratin materials, comprising a physiologically acceptable medium containing at least one liquid fatty phase, a colloidal dispersion of particles that are solid at ambient temperature and a dispersion of polymer particles that are surface-stabilized in the said liquid fatty phase with a stabilizer.

2. Composition according to claim 1, characterized in that the particles of the colloidal dispersion are particles chosen from pigments, nacres and fillers, and mixtures thereof.

3. Composition according to claim 1 or 2, characterized in that the particles of the colloidal dispersion contain coloured particles.

4. Composition according to one of the preceding claims, characterized in that the colloidal dispersion represents from 0.5% to 60% by weight of the composition and better still from 2% to 40% and even better still from 2% to 50%.

5. Composition according to one of the preceding claims, characterized in that the colloidal dispersion comprises a dispersant for the particles.

6. Composition according to claim 5, characterized in that the dispersant represents from 0.3 to 5 mg/m² and preferably from 0.5 to 4 mg/m² of surface area of particles.

7. Composition according to claim 5 or 6, characterized in that the dispersant is chosen from poly(12-hydroxystearic acid) stearate, poly(12-hydroxystearic acid) and diglyceryl 2-dipolyhydroxystearate, and mixtures thereof.

8. Composition according to one of the preceding claims, characterized in that the colloidal dispersion contains a fatty substance that is liquid at ambient temperature, forming part of the liquid fatty phase.

9. Composition according to one of the preceding claims, characterized in that the polymer in dispersion can form a film.

10. Composition according to one of the preceding claims, in which at least one ingredient chosen from cosmetic and dermatological active agents, and mixtures thereof, is provided.

11. Composition according to one of the preceding claims, in which the polymer in dispersion is chosen from free-radical polymers, polycondensates and polymers of natural origin, and mixtures thereof.

12. Composition according to one of the preceding claims, in which the polymer in dispersion is chosen from polyurethanes, polyurethane-acrylics, polyureas, polyurea/polyurethanes, polyester-polyurethanes, polyether-polyurethanes, polyesters, polyesteramides, fatty-chain polyesters, alkyds; acrylic and/or vinyl polymers or copolymers; acrylic-silicone copolymers; polyacrylamides; silicone polymers, fluoro polymers, and mixtures thereof.

13. Composition according to one of the preceding claims, in which the liquid fatty phase consists of oils of mineral, animal, plant or synthetic origin, carbon-based, hydrocarbon-based, fluoro and/or silicone oils, alone or as a mixture.

14. Composition according to one of the preceding claims, in which the liquid fatty phase is chosen from liquid paraffin, liquid petroleum jelly, volatile or non-volatile isoparaffins, mink oil, turtle oil, soybean oil, perhydrosqualene, sweet almond oil, beauty-leaf oil, palm oil, Parleam oil, grapeseed oil, sesame oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; myristic acid, palmitic acid, stearic acid, behenic acid, oleic acid, linoleic acid, linolenic acid or isostearic acid; stearyl alcohol, oleyl alcohol, linoleyl alcohol, linolenyl alcohol, isostearyl alcohol or octyldodecanol; volatile or non-volatile silicone oils of PDMS type that are optionally phenylated or optionally substituted with aliphatic and/or aromatic groups, or with functional groups; polysiloxanes modified with fatty acids, fatty alcohols or polyoxyalkylenes; fluorosilicones and perfluoro oils and mixtures thereof.

15. Composition according to one of the preceding claims, characterized in that the fatty phase contains at least one oil that is not volatile at ambient temperature and atmospheric pressure.

16. Composition according to one of the preceding claims, in which the stabilizer is chosen from block polymers, grafted polymers and random polymers, and mixtures thereof.

17. Composition according to one of the preceding claims, in which the stabilizer is chosen from silicone polymers grafted with a hydrocarbon-based chain; hydrocarbon-based polymers grafted with a silicone chain; grafted copolymers having an insoluble skeleton of polyacrylic type with soluble grafts of poly(12-hydroxystearic acid) type; grafted-block or block copolymers comprising at least one block of polyorganosiloxane type and at least one block of a free-radical polymer; grafted-block or block copolymers comprising at least one block of polyorganosiloxane type and at least [lacuna] of a polyether; copolymers of acrylates or methacrylates of a C₁-C₄ alkyl, or of acrylates or methacrylates of a C₈-C₃₀ alkyl; grafted-block or block copolymers comprising at least one block resulting from the polymerization of ethylenic monomers optionally comprising conjugated bonds and at least one block of a vinyl polymer; grafted-block or block copolymers comprising at least one block resulting from the polymerization of ethylenic monomers optionally comprising conjugated bonds and at least one block of an acrylic polymer; grafted-block or block copolymers comprising at least one block resulting from the polymerization of diene and at least one block of a polyether, and mixtures thereof.

18. Composition according to one of the preceding claims, characterized in that the stabilizer is a grafted-block or block polymer, comprising at least one block resulting from the polymerization of diene and at least one block of a vinyl polymer.

19. Composition according to one of the preceding claims, also comprising at least one additional fatty phase chosen from waxes, gums and/or pasty fatty substances, that are hydrocarbon-based, silicon-based and/or fluorinated, of plant, animal, mineral or synthetic origina, and mixtures thereof.

20. Composition according to one of the preceding claims, characterized in that the polymer in dispersion represents (as solids) up to 60% of the total weight of the composition.

21. Composition according to one of the preceding claims, characterized in that the polymer in dispersion represents (as solids) from 2% to 60% of the total weight of the composition, and preferably from 4 to 2.5.

22. Composition according to one of the preceding claims, which is in the form of a stick or tube, in the form of a soft paste, in the form of a dish, an oily gel, an oily liquid, a vesicular dispersion containing ionic and/or nonionic lipids, or a water-in-oil, oil-in-water or multiple emulsion.

23. Composition according to one of the preceding claims, which is in anhydrous form.

24. Composition according to one of the preceding claims, which is in the form of a care and/or make-up product for the skin and/or for the lips.

25. Composition according to one of the preceding claims, which is in the form of a foundation, a face powder, an eyeshadow, a lipstick, a lipcare balm or base, a concealer product, an eyeliner or a mascara.

26. Cosmetic care process or make-up process for the lips or the skin, which consists in applying a cosmetic composition as defined in the preceding claims to the lips or the skin, respectively.

27. Process for limiting the migration of a make-up composition or care composition for the skin or the lips and/or to increase the staying power over time of this composition and/or its stability, containing a liquid fatty phase and at least one ingredient chosen from dyestuffs in the form of coloured particles that are solid at ambient temperature, which consists in introducing into the liquid fatty phase polymer particles that are dispersible in the liquid fatty phase and surface-stabilized with a stabilizer, and in introducing into the said liquid fatty phase the said coloured particles in the form of a colloidal dispersion.

28. Use, in a composition for application to the skin, the lips and integuments, of particles of at least one polymer that are dispersed in a liquid fatty phase and surface-stabilized with a stabilizer, and of a colloidal dispersion of particles that are solid at ambient temperature, and especially coloured, to limit the migration of the composition and/or to increase its staying power over time and/or to obtain a uniform make-up effect.

29. Use, in a composition for application to the skin, the lips and integuments, of particles of at least one polymer that are dispersed in a liquid fatty phase and surface-stabilized with a stabilizer, and of a colloidal dispersion of particles that are solid at ambient temperature, and especially coloured, to conserve the gloss of the said composition.

30. Process for manufacturing a stable composition for application to keratin materials, which consists in introducing into a physiologically acceptable liquid medium a) a dispersion of polymer particles that are surface-stabilized in a liquid fatty phase with a stabilizer, b) a colloidal dispersion of particles that are solid at ambient temperature, chosen from pigments, nacres and fillers and mixtures thereof, and in mixing the said medium to which is added the said dispersions a) and b).

31. Use of a colloidal dispersion of particles that are solid at ambient temperature, chosen from pigments, nacres and fillers, and mixtures thereof, in a composition for application to keratin materials, containing a dispersion of polymer particles that are surface-stabilized in a liquid fatty phase with a stabilizer, to stabilize the said composition and/or to give it a uniform appearance.
